(19) United States
(12) Patent Application Publication
(10) Pub. No.: US 2003/0149285 A1
(43) Pub. Date: Aug. 7, 2003

(54) Method for producing 1,1' binaphthalenylidene-4,4'-diones

(76) Inventors: Michael Hauck, Naters (CH); Josef Heveling, Hartbeespoort (ZA); Alain Wellig, Ried-Morel (CH)

Correspondence Address: Fisher Christen & Sabol, Suite 1108, 1725 K Street N W, Washington, DC, 20006, US

(21) Appl. No.: 10/221,868

(22) PCT Filed: Mar. 16, 2001
(86) PCT No.: PCT/EP01/03045

(30) Foreign Application Priority Data
Mar. 17, 2000 (EP) 00105700.9

(51) Int. Cl. C07C 46/04

(52) U.S. Cl. 552/295

(57) ABSTRACT

The invention relates to a method for producing 1,1′ binaphthalenylidene-4,4′-diones of general formula (I), wherein R¹ represents C₁₋₆ alkyl, C₁₋₆ alkoxy, phenyl, substituted phenyl, benzyl or benzyloxy; R², R³, R⁴, and R⁵ independently of one another represent hydrogen, C₁₋₆ alkyl, C₁₋₆ alkoxy, phenyl, substituted phenyl, benzyl or benzyloxy. Production takes place by oxidative coupling of the corresponding naphthols in the presence of a peroxide and a precious metal catalyst.

The present invention relates to a process for preparing 1,1′-binaphthalenylidene-4,4′-diones.

1,1′-Binaphthalenylidene-4,4′-diones may be prepared, for example, by oxidative coupling of 1-naphthols in the presence of silver oxide. An appropriate preparative process is described by A. Kral et al. in Z. Naturforsch. B 1993, 48, 1401-1407.

The known processes are not catalytic processes. This leads to a high consumption of usually heavy metal oxidants which have to be either regenerated or disposed of in an environmentally responsible manner at additional cost.

It is accordingly an object of the present invention to provide a process which does not have the abovementioned disadvantages.

According to the invention, this object is achieved by the process as claimed in claim 1.

It has been found that 1,1′-binaphthalenylidene-4,4′-diones of the general formula

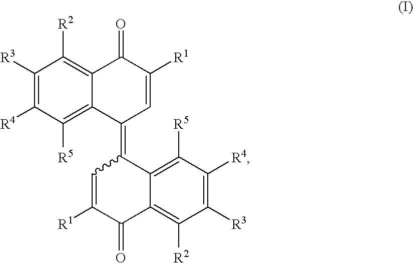

where

R¹ is C₁₋₆-alkyl, C₁₋₆-alkoxy, phenyl, substituted phenyl, benzyl or benzyloxy;

R², R³, R⁴ and R⁵ are each independently hydrogen, C₁₋₆-alkyl, C₁₋₆-alkoxy, phenyl, substituted phenyl, benzyl or benzyloxy, may be prepared by oxidative coupling of naphthols of the general formula

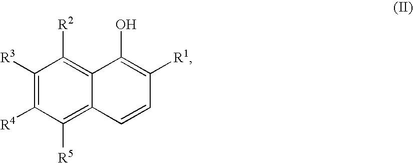

where R¹, R², R³, R⁴ and R⁵ are each as defined above,

in the presence of a peroxide and also of a noble metal catalyst.

The R¹ radical is preferably methyl. The R², R³, R⁴ and R⁵ radicals are each preferably hydrogen.

The compounds of the formula I may occur either in the (E) form or else in the (Z) form. Preference is given to the (E) form.

C₁₋₆-alkyl is hereinbelow any linear or branched alkyl group having 1-6 carbon atoms, for example methyl, ethyl, propyl, isopropyl, butyl, isobutyl, sec-butyl, tert-butyl, pentyl, isopentyl, tert-pentyl, neopentyl, hexyl or isohexyl.

C₁₋₆-alkoxy is hereinbelow any linear or branched alkoxy group having 1-6 carbon atoms, for example methoxy, ethoxy, propoxy, isopropoxy, butoxy, isobutoxy, sec-butoxy, tert-butoxy, pentyloxy, isopentyloxy, tert-pentyloxy, neopentyloxy, hexyloxy or isohexyloxy.

The phenyl radical may also carry one or more identical or different substituents in the ortho-, meta- or para-positions. Examples of useful substituents include halogens such as fluorine, chlorine, bromine or iodine, C₁₋₆-alkyl, halogenated C₁₋₆-alkyl, for example trifluoromethyl, and C₁₋₆-alkoxy. Examples of substituted phenyl radicals include methylphenyl, dimethylphenyl, ethylphenyl, propylphenyl, methoxyphenyl, ethoxyphenyl, propoxyphenyl and the like.

The naphthols of the formula II are known compounds or are preparable in a similar manner to known compounds. 2-Alkyl-1-naphthols are easily obtainable by reduction of the appropriate aryl alkyl ketones or by alkylation. 2-Alkoxy-1-naphthols can be synthesized, for example, starting from the appropriate 2-alkoxy-naphthalene-1-carbaldehydes, as described in the above-cited literature.

A preferred naphthol is 2-methyl-1-naphthol.

Peroxides include both organic and inorganic peroxides. Examples of suitable peroxides include hydrogen peroxide, perbenzoic acid and peracetic acid. Preference is given to hydrogen peroxide, advantageously as an aqueous 10-30% solution. Particular preference is given to a 30% aqueous solution of hydrogen peroxide.

Useful noble metal catalysts are in particular platinum, rhodium or ruthenium catalysts. Preference is given to a platinum catalyst.

Advantageously, the noble metal catalysts are used in combination with other metals, for example bismuth, lead or cerium. Preference is given to a combination of platinum and bismuth.

The catalysts may be used unsupported or applied to a suitable support material. Preference is given to using supported catalysts. Useful supports include all conventional support materials, for example activated carbon, alumina, silica, silica-alumina, silicon carbide, titanium dioxide, magnesium oxide or zeolites. Particular preference is given to activated carbon.

The support materials advantageously contain 0.1-30% by weight, preferably 0.5-10% by weight, of metal.

Particular preference is given to a platinum/bismuth catalyst on activated carbon, for example having 5% of Pt and 5% of Bi.

These catalysts are commercially obtainable, for example from Degussa or Heraeus.

The process according to the invention is advantageously carried out at a temperature of 20-120° C., preferably of 50-100° C.

The process according to the invention is advantageously carried out in a suitable solvent. Useful solvents are in particular lower alcohols such as methanol, ethanol, propanol, isopropanol or mixtures of alcohols with acetic acid.

The compounds of the formula I absorb visible light in the range from 480 nm to 560 nm and are therefore suitable for use as dyes, as described in the above-cited literature.

The example hereinbelow illustrates the performance of the process according to the invention without restricting it.

### EXAMPLE

### 3,3′-Dimethyl-1,1′-binaphthalenylidene-4,4′-dione

5 g (31.6 mmol) of 2-methyl-1-naphthol, 50 g of methanol and 0.4 g of a catalyst having 5% of Pt and 5% of Bi on activated carbon (Degussa) were initially charged in a 100 ml three-necked flask. The reaction mixture was heated in an oil bath at 60° C. with stirring. Within one hour, 8 g of a 30% aqueous H₂O₂ solution was added dropwise to the solution (70.6 mmol of H₂O₂). During the reaction, a red solid precipitated out. In order to ensure stirrability, a further 15 ml of methanol were added 25 min after the beginning of the dropwise addition. After a further 5 min, 10 ml of acetic acid (conc.) were added. At the end of the H₂O₂ addition, an intensively red-colored suspension was obtained. This was cooled, then filtered through a G4 suction filter. The filtercake was washed with 20 ml of methanol. After concentration by evaporation, the filtrate gave 0.8 g of a dark red resin (fraction 1). The filtercake was flurried successively with acetone, methylene chloride and then with acetone again and filtered in each case. The collected filtrates were concentrated to dryness; 0.8 g of a dark red solid was obtained (fraction 2). The filtercake was then dissolved in 1.5 l of hot acetone. The solution was freed of catalyst using a G4 suction filter into which a 2 cm thick celite layer had additionally been introduced. The filter layer was washed with 40 ml of hot acetone. When the acetone solution was concentrated (60° C./450 mbar) to a volume of about 200 ml, a red solid crystallized out. The mixture was admixed with 150 ml of methanol and concentrated further to a total volume of about 150 ml. The mixture was then cooled to 25° C. and filtered. After drying (50° C./25 mbar), fraction 3 was obtained (3.5 g). Concentration of the filtrate gave a further fraction 4 (0.3 g).

The red solid was characterized by elemental analysis, UV, IR and MS, supplemented by ¹H NMR and ¹³C NMR measurements, and identified as 3,3′-dimethyl-1,1′-binaphthalenylidene-4,4′-dione.

The purity of the fraction 3 obtained by the above-described procedure was estimated by means of NMR to be about 80%.

In total, 5.4 g of solid were obtained. Based on a content of 80%, the yield is 87.5%.

What is claimed is:

1. A process for preparing 1,1′-binaphthalenylidene-4,4′-diones of the general formula 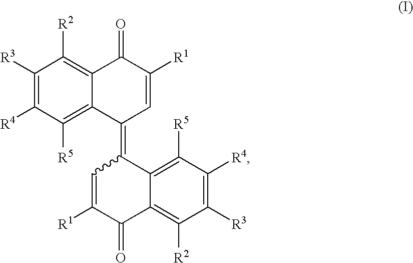
where
R¹ is C₁₋₆-alkyl, C₁₋₆-alkoxy, phenyl, substituted phenyl, benzyl or benzyloxy;
R², R³, R⁴ and R⁵ are each independently hydrogen, C₁₋₆-alkyl, C₁₋₆-alkoxy, phenyl, substituted phenyl, benzyl or benzyloxy,
by oxidative coupling of naphthols of the general formula 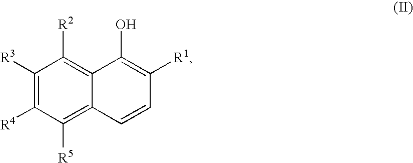
where R¹, R², R³, R⁴ and R⁵ are each as defined above,
characterized in that the oxidative coupling is effected in the presence of a peroxide and also of a noble metal catalyst.

2. The process as claimed in claim 1, where R¹ is methyl and R², R³, R⁴ and R⁵ are each hydrogen.

3. The process as claimed in claim 1 or 2, characterized in that the peroxide used is hydrogen peroxide, in particular as an aqueous 10-30% solution.

4. The process as claimed in any of claims 1 to 3, characterized in that the noble metal catalyst used is a platinum, rhodium or ruthenium catalyst, optionally in combination with bismuth, lead or cerium.

5. The process as claimed in claim 4, characterized in that the catalyst is applied to a support material.

6. The process as claimed in claim 5, characterized in that the catalyst is a Pt/Bi catalyst on an activated carbon support.

7. The process as claimed in any of claims 1 to 6, characterized in that the oxidative coupling is carried out at a temperature of 20-120° C., preferably 50-100° C.

8. The process as claimed in any of claims 1 to 7, characterized in that the oxidative coupling is carried out in an alcohol or an alcohol/acetic acid mixture.
